(19) United States
(12) Patent Application Publication
(10) Pub. No.: US 2019/0029288 A1
(43) Pub. Date: Jan. 31, 2019

(54) CACAO-DERIVED WATER EXTRACT, FOOD AND DRINK CONTAINING SAME, METHOD FOR MANUFACTURING CACAO EXTRACT, AND METHOD FOR EXTRACTING POLYPHENOL

(71) Applicant: MEIJI CO.,LTD., Chuo-ku, Tokyo (JP)

(72) Inventors: Kazuji TAMURA, Odawara-shi, Kanagawa (JP); Asami TSUCHIYA, Odawara-shi, Kanagawa (JP); Nobutaka YAHIRO, Odawara-shi, Kanagawa (JP)

(21) Appl. No.: 16/072,726

(22) PCT Filed: Feb. 27, 2017
(86) PCT No.: PCT/JP2017/007341

§ 371 Date: Jul. 25, 2018

(30) Foreign Application Priority Data
Feb. 29, 2016 (JP) 2016-037005

(51) Int. Cl. A23G 1/02; A23L 33/105

(52) U.S. Cl. CPC A23G 1/02; A23L 33/105; A23V 2002/00; A23V 2300/31; A23V 2250/21166; A23V 2250/2132; A23V 2300/14; A23V 2200/02

(57) ABSTRACT

Provided is cacao extract with better flavor and in which miscellaneous flavor is reduced, and a method for producing such cacao extract. The cacao-derived water extract of the present invention contains polyphenol. In this cacao water extract, the ratio of a total mass of dimeric to tetrameric proanthocyanidins to a total mass of monomeric to octameric proanthocyanidins is 40% or more. Also, in this method for producing cacao extract, an active ingredient such as polyphenol is extracted from a cacao raw material such as cacao powder using water adjusted to a temperature within a range from 50° C. to 90° C. inclusive and to a pH within a range from 2.0 to 5.0 inclusive.

### TECHNICAL FIELD

The present invention relates to a cacao-derived water extract, foods or drinks containing the same, a method for producing a cacao extract, and a method for extracting polyphenol.

### BACKGROUND ART

In recent years, the antioxidant function of polyphenols has attracted attention and has been widely used as an active ingredient of functional foods. Examples of plant raw materials containing a large amount of polyphenols include cacaos, green tea, and apples. Cacaos are known to contain low molecular proanthocyanidins having high physiological activity among polyphenols.

Thus, cacaos are useful as a raw material for antioxidant compositions, and various methods for extracting proanthocyanidins from cacaos have been studied. For example, Patent Document 1 discloses a method of extracting polyphenols from cacao solids using an organic solvent such as methyl acetate, ethyl acetate or the like.

### CITATION LIST

Patent Document 1: Japanese Unexamined Patent Application Publication No. 2012-211156

### SUMMARY OF THE INVENTION

Problem to be Solved by Invention

However, when extraction is carried out using an organic solvent, the flavor is deteriorated, so that the application to foods which emphasize palatability is restricted.

In view of the above, it is an object of the present invention to provide a cacao extract having better flavor while suppressing miscellaneous flavor, and a method for producing such a cacao extract.

### Means for Solving the Problem

In view of the above problems, the inventors of the present application conducted intensive studies to obtain a cacao extract from cacao raw material using water as an extraction solvent instead of an organic solvent to prevent a deterioration of the flavor of the obtained extract. It was found that a cacao extract containing a large amount of polyphenols having high physiological activity (particularly, proanthocyanidins among them) can be obtained by adjusting the temperature and pH of water used for extraction within an appropriate range, leading to completion of the present invention.

The cacao-derived water extract according to the present invention contains a polyphenol. This polyphenol includes proanthocyanidins In the cacao-derived water extract according to the present invention, the ratio of the total mass of dimeric proanthocyanidins, trimeric proanthocyanidins, and tetrameric proanthocyanidins to the total mass of monomeric to octameric proanthocyanidins is 40% or more.

In the above-described cacao-derived water extract, the ratio of the total mass of the dimer proanthocyanidins, the trimer proanthocyanidins, and the tetramer proanthocyanidins to the total mass of monomeric to octameric proanthocyanidins is preferably 51% or more.

It is preferable that the cacao-derived water extract contains procyanidin B2 and catechin as the polyphenol, and the ratio of the mass of the procyanindin B2 to the mass of the catechin is within a range from 5 to 20 inclusive.

It is preferable that, in the cacao-derived water extract, the polyphenol is substantially free of octameric proanthocyanidins.

It is preferable that the cacao-derived water extract further contains theobromine with a concentration within a range from 5 mg/g to 20 mg/g inclusive.

In addition, the food or drink according to the present invention contains any one of the above-described cacao water extracts. However, the food or drink according to the present invention does not include animals and plants per se.

Also, the method for producing a cacao extract according to the present invention includes providing a cacao or a cacao processed product as a raw material and extracting a cacao-derived component using water adjusted to have a temperature within a range from 50° C. to 90° C. inclusive and a pH within a range from 2.0 to 5.0 inclusive.

It is preferable that the method for producing the cacao extract includes adjusting the pH of the water to a pH that is greater than 4.0 and lower than or equal to 5.0.

It is preferable that in the method for producing the cacao extract, the raw material is roughly cmshed cacao beans.

Another cacao extract according to the present invention is made from cacao or cacao processed product. The cacao extract according to the present invention is extracted using water having a temperature within a range from 50° C. to90° C. inclusive and a pH within a range from 2.0 to 5.0 inclusive.

In addition, a method of extracting polyphenol according to the present invention includes immersing cmshed cacao raw material in water having a temperature within a range from 50° C. to 90° C. inclusive and a pH within a range from 2.0 to 5.0 inclusive to extract a cacao-derived component in the water, and extracting polyphenol in the water. This method for extracting polyphenols extracts polyphenol in the water while maintaining an extracted amount of miscellaneous flavor components to a low level.

An antioxidant composition according to another aspect of the present invention contains cacao-derived polyphenol as an active ingredient, and a ratio of a total mass of dimeric proanthocyanidins, trimeric proanthocyanidins and tetrameric proanthocyanidins to a total mass of monomeric to octameric proanthocyanidins is 40% or more. Also, a food or drink (excluding animals and plants per se), functional foods, functional drinks, pharmaceuticals, cosmetics, and chemical products according to another aspect of the present invention contain the antioxidant composition.

### Effect of the Invention

As described above, the cacao-derived water extract of the present invention contains a large amount of proanthocyanidins ranging from dimers to tetramers, which are more physiologically active among polyphenols. In addition, the cacao-derived water extract of the present invention is extracted using water, and thus it has less miscellaneous flavor components and has a good flavor compared with extracts extracted using an organic solvent. Thus, for example, it can be effectively used for foods and beverages having antioxidant function, pharmaceuticals or the like.

Further, the process for producing a cacao extract of the present invention allows for producing a cacao extract containing polyphenols at a relatively high content by using water as an extraction solvent. In addition, the method for producing a cacao extract of the present invention allows for producing a cacao extract having less miscellaneous flavor components compared with a case of using an organic solvent.

Further, the polyphenol extraction method of the present invention allows for obtaining an extract containing polyphenols in a state with less miscellaneous flavor components as compared with a case of using an organic solvent as an extraction solvent.

### DESCRIPTION OF EMBODIMENTS

The present invention will now be described in more detail. However, the present invention should not be limited to this.

[Method for Producing Cacao Extract]

A method for producing a cacao extract according to the present invention will now be described. The method for producing a cacao extract of the present invention extracts cacao-derived components, with a cacao or a cacao processed product as a raw material, using water adjusted to a temperature within a range of from 50° C. to 90° C. inclusive and a pH within a range of from 2.0 to 5.0 inclusive. The cacao-derived extract of the present invention can also be produced by this method. However, the method for producing the cacao-derived extract of the present invention should not be necessarily limited to this method.

The cacao raw material used in the production method of the present invention is a cacao or cacao processed product. Cacao contains unfermented cacao beans. Cacao processed products include cacao beans after fermentation, unfermented cacao ground products (unfermented cacao powder), ground cacao products after fermentation (fermented cacao powder), cacao nibs, cacao masses and the like. Note that the origin and the growth situation of the cacao beans to be used should not be particularly limited.

Unfermented cacao powder contains more physiologically active ingredients such as antioxidant ingredients than cacao powder after fermentation. Thus, it is preferable that the production method of the present invention uses unfermented cacao powder as a raw material. Alternatively, it is preferable that the production method of the present invention uses roughly crushed cacao beans as a raw material. Here, the roughly crushed cacao beans refer to one having a particle diameter within a range from about 1.0 to about 4.75 mm inclusive. Note that the particle size of the roughly crushed cacao beans can be determined by a method of stepwise sifting with a sieve having different mesh size. In other words, the particle size of roughly crushed cacao beans within the range from about 1.0 mm to about 4.75 mm inclusive means that such beans do not pass through a sieve having a mesh size of less than 1.0 mm and pass through a sieve having a mesh size of greater than 4.75 mm.

Further, it is preferable that the roughly crushed cacao beans include pulverized materials having a particle size (maximum diameter) of 1.0 mm or more with a weight ratio of 70% or more; it is more preferable that they include pulverized materials having a particle size (maximum diameter) of 1.0 mm or more with a weight ratio of 80% or more. Producing the cacao extract using such crushed cacao beans makes solid-liquid separation easier, thus not only enhancing production efficiency but also obtaining cacao extract containing low-molecular proanthocyanidins with a greater ratio.

Also, the production method of the present invention extracts cacao-derived components using water adjusted to a temperature within a range from 50° C. to 90° C. inclusive and a pH within a range from 2.0 to 5.0 inclusive. To extract cacao-derived components, the production method of the present invention uses only water as an extraction solvent.

In the present specification, “using only water as an extraction solvent” means that a solvent containing water (H₂O) as a main component is used as a solvent to be firstly added to a cacao raw material such as cacao powder. Water to be used includes pure water, distilled water, ion exchanged water, tap water and the like which generally fall within the category of water. Note that for example, a case in which a pH adjuster such as citric acid is added to water in order to adjust the pH of the extraction solvent is also included in the case of “using only water as an extraction solvent”. In addition, after adding water to cacao raw material and dispersing the cacao raw material in water, a pH adjuster such as citric acid or the like may be added later in order to adjust the pH and the like.

In addition, in the production method of the present invention, the temperature of water is adjusted to a temperature within a range from 50° C. to 90° C. inclusive. Adjusting the temperature of water to a temperature within a range from 50° C. to 90° C. inclusive means, for example, that after adding room temperature water to the cacao raw material, cacao suspension water is heated to a temperature within a range from 50° C. to 90° C. inclusive. In the production method of the present invention, the temperature of water added to the cacao raw material may be previously heated to a temperature within a range from 50° C. to 90° C. inclusive.

Setting the temperature of water to 50° C. or higher allows an extraction amount of polyphenol to be increased. In addition, the temperature of water is preferably within a range from 65° C. to 90° C. inclusive. This makes it possible to obtain cacao extract with higher content of procyanidin B2.

In the production method of the present invention, the pH of water is adjusted to a pH within a range from 2.0 to 5.0 inclusive. Adjusting the pH of water within a range from 2.0 to 5.0 inclusive means that, after adding neutral (approximately pH 7.0) water to the cacao raw material, the pH of the cacao suspension water is adjusted to a pH within a range from 2.0 to 5.0 inclusive. Citric acid (anhydrous citric acid powder, saturated citric acid solution, or the like), malic acid, tartaric acid, lactic acid, phosphoric acid, hydrochloric acid, sulfuric acid and the like can be used for adjusting the pH as above. In the production method of the present invention, the pH of water to be added to the cacao raw material may be previously adjusted to a pH within a range from 2.0 to 5.0 inclusive.

Adjusting the pH of water to 5.0 or less allows the extraction amount of polyphenol to be increased. In addition, increasing the pH of water to 2.0 or more suppresses an increase in acidity of the obtained extract. In addition, when the obtained extract is a liquid, maintaining the pH of the extract in the acidic region stabilizes polyphenols such as proanthocyanidins.

In addition, it is preferable that the pH of water is adjusted to exceed 4.0. As described above, adjusting the pH of water to be included within a range closer to neutrality makes it possible to efficiently prevent an increase in acidity of the obtained extract.

Note that the pH of water can be set to 4.0 or less. Making the pH of water more acidic in this manner makes it possible to obtain a cacao extract with higher polyphenol content. Further, the ratio of the total mass of the proanthocyanidins from the dimer to the tetramer to the total mass of the proanthocyanidins from the monomer to the octamer can be further increased.

The amount of water to be used is preferably within a range from 5 to 100 inclusive in the mass ratio of water to cacao material; it is more preferably within a range from 8 to 25 inclusive in the mass ratio of water to cacao material.

In the production method according to one embodiment, it is preferable to stir cacao suspension water for a certain period of time (for example, a period within a range from 5 minutes to 5 hours inclusive) in a state in which the above-described temperature and pH are maintained This makes it possible to more efficiently extract the active ingredient from the cacao raw material. For example, setting the extraction time to 5 minutes or more makes it possible to obtain a cacao extract containing more proanthocyanidins Note that it is more preferable that the extraction time (a time period for stirring while heating the cacao suspension water) is within a range from 1 hour to 2 hours inclusive in order to inhibit the denaturation and decomposition of the proanthocyanidins in the extract.

Further, in the production method according to one embodiment, a solid-liquid separation step may be included after the stirring step of the cacao suspension water. For solid-liquid separation, methods such as centrifugal separation and filtration can be used. Either the centrifugation or the filtration may be carried out; otherwise, both of the centrifugation and the filtration may be carried out. Conditions for centrifugation should not be limited; however, the centrifugal force may be set within a range from 2,000 g to 12,000 g, and the required time may be set within a range from 5 minutes to 45 minutes inclusive, for example. Solid liquid separation can be performed in a shorter time by setting the centrifugal force to 2,000 g or more. In addition, setting the centrifugal force to 12,000 g or less makes it possible to obtain the active ingredient contained in the supernatant in a stable state. Also, setting the required time to 5 minutes or more makes it possible to reduce the amount of miscellaneous flavor components contained in the supernatant. Further, setting the required time to 45 minutes or less makes it possible to shorten the time for producing the extract. The time required for centrifugal separation is more preferably within a range from 5 minutes to 30 minutes inclusive, and further preferably within a range from 5 minutes to 15 minutes inclusive. For filtration, a strainer having a mesh size within a range from 50 mesh to 200 mesh inclusive can be used. Instead of a strainer, filter cloth or filter paper can also be used.

The supernatant obtained by the above-described solid-liquid separation step is then concentrated. For concentration, a vacuum concentration apparatus such as a rotary evaporator can be used. In this way, cacao extract is obtained.

Note that in the producing method according to another embodiment, the above-described extraction step and solid-liquid separation step may be performed a plurality of times. More specifically, water whose amount is equal to the amount of the obtained supernatant is added to the residue obtained by the above-described solid-liquid separation step, for example; subsequently, after stirring the residue again by suspension, the supernatant may be recovered by centrifugation or the like. As described above, performing the extraction step and the solid-liquid separation step a plurality of times makes it possible to increase the extraction rate of active ingredients such as polyphenol in the obtained extract.

The cacao extract produced by using the production method of the present invention is also included in the technical scope of the present invention. In other words, the cacao extract according to the present invention is one which is extracted from a cacao or cacao processed product as a raw material using water having a temperature within a range from 50° C. to 90° C. inclusive and a pH within a range from 2.0 to 5.0 inclusive.

[Cacao-Derived Water Extract]

Next, the cacao-derived water extract according to the present invention will be described. The cacao-derived water extract according to the present invention is extracted from a cacao as a raw material using water as an extraction solvent. The category of water includes a solvent containing water as a main component, an aqueous solution containing a substance soluble in water, and the like. Among them, the cacao-derived water extract according to the present embodiment is preferably produced using only water as an extraction solvent. This makes it possible to prevent a deterioration of the flavor of the extract as compared with extract using an organic solvent such as ethyl acetate, methyl acetate, and alcohols (ethanol or the like) as an extraction solvent. Furthermore, using water as an extraction solvent makes it possible to reduce the miscellaneous flavor components contained in the extract.

The cacao-derived water extract of the present invention contains polyphenol as an active ingredient. Polyphenols have long been known to have antioxidant function. The antioxidant function of polyphenols contributes to the reduction of active oxygen harmful to living bodies. For this reason, many health foods, functional foods, cosmetics and the like containing polyphenol as an active ingredient are on the market in recent years.

Polyphenols include monomer catechins, proanthocyanidins, which are catechin polymers, and the like. The cacao-derived water extract of the present invention significantly contains a large amount of proanthocyanidins among polyphenols. It is known that the functions of catechin molecules from the dimer to the tetramer (i.e., proanthocyanidins from the dimers to the tetramers inclusive) are significantly high among proanthocyanidins (Patent Document: WO 2006/090830).

The cacao-derived water extract according to the present invention contains a relatively large amount of proanthocyanidins from the dimers to the tetramers inclusive with a higher function. More specifically, in the cacao-derived water extract of the present invention, the ratio of the total mass of dimeric proanthocyanidins, trimeric proanthocyanidins, and tetrameric proanthocyanidins (dimeric to tetrameric proanthocyanidins) to the total mass of the proanthocyanidins from the monomer to the octamer (monomeric to octameric proanthocyanidins) is 40% or more. The ratio of the total mass of the dimeric to tetrameric proanthocyanidins to the total mass of the monomeric to octameric proanthocyanidins is preferably 44% or more, more preferably 50% or more; furthermore, it is more preferably 51% or more, still more preferably 54% or more. This enhances the antioxidant function of the cacao-derived water extract of the present invention.

The upper limit of the content ratio of the proanthocyanidins from the dimers to the tetramers inclusive should not be particularly limited; however, it may be, for example, 70% or less, preferably 60% or less.

The polyphenol contained in the cacao-derived water extract of the present invention contains a relatively large amount of procyanidin B2. Since procyanidin B2 inhibits the activity of protein kinase, and thus it is used as an active ingredient of inflammation inhibitor and apoptosis inhibitor. Procyanidin B2 is a type of polyphenol; more specifically, it has a chemical structure as shown in the following structural formula (A).

### [Chemical Formula 1]

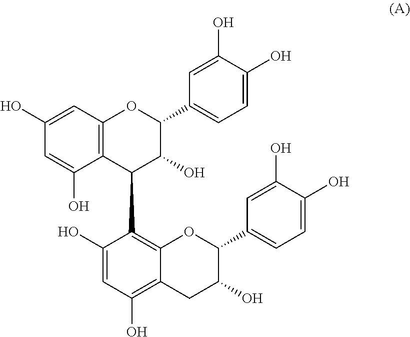 [SMILES: Oc1cc(O)c2c(c1)O[C@H](c1ccc(O)c(O)c1)[C@H](O)[C@H]2c1c(O)cc(O)c2c1O[C@H](c1ccc(O)c(O)c1)[C@H](O)C2]

The cacao-derived water extract of the present invention includes procyanidin B2 and catechin as polyphenols. In the cacao-derived water extract of the present invention, it is preferable that the ratio of the mass of procyanidin B2 to the mass of catechin is within a range from 5 to 20 inclusive. In the cacao-derived water extract of the present invention, it is more preferable that the ratio of the mass of procyanidin B2 to the mass of catechin is within a range from 10 to 20 inclusive. As described above, in the cacao-derived water extract of the present invention, it is possible to contain more procyanidin B2, which is more physiologically active, relative to catechin that is monomeric proanthocyanidins.

Further, in the cacao-derived water extract of the present invention, it is preferable that the content of proanthocyanidins of a multimer having a larger molecular weight is small. More specifically, it is preferable that the cacao-derived water extract of the present invention does not contain octameric proanthocyanidins. This makes it possible to increase the content ratio of dimeric to tetrameric proanthocyanidins having higher physiological activity. In addition, the multimer (polymer) of catechin corresponding to multimeric proanthocyanidins has strong bitterness and astringency. Thus, reducing the content of multimeric proanthocyanidins makes it possible to reduce the bitterness and astringency of the cacao-derived water extract.

The cacao-derived water extract of the present invention may contain theobromine with a concentration within a range from 5 mg/g to 20 mg/g inclusive. The concentration of theobromine is, for example, a concentration in the extract after concentration of the supernatant obtained in the solid-liquid separation step of the above-described production method. Theobromine is a xanthine alkaloid that is relatively abundant in cacao. Although theobromine has a physiological activity such as a vasodilating effect and a diuretic effect, it also causes an unpleasant flavor such as bitterness. Thus, it is desirable to contain it in an appropriate amount in the cacao extract. For example, using the above-described method for producing cacao extract of the present invention allows the concentration of theobromine to be within an appropriate range from 5 mg/g to 20 mg/g inclusive. Further, using the above-described method allows the concentration of theobromine to be within a preferable range from 5 mg/g to 10 mg/g inclusive.

The cacao-derived water extract of the present invention is preferably distributed in the form of an aqueous solution containing no organic solvent. This makes it possible to prevent a flavor deterioration of the extract. More preferably, the cacao-derived water extract of the present invention is in the form of a concentrated aqueous solution.

Further, the cacao-derived water extract of the present invention may be concentrated and then powdered. As a method for pulverizing the concentrated extract, a spray drying method, a freeze drying method, a pressure reduced drying method, or the like can be used. To maintain the flavor of the cacao-derived water extract of the present invention in a satisfactory state, it is preferable to distribute it in a liquid state such as an extract solution or an aqueous solution. Conversely, to maintain the active ingredient in a stabilized state, powdering is preferable.

[Usage of Cacao-Derived Water Extract of the Present Invention]

Polyphenol is contained, as an antioxidant component, in the cacao-derived water extract of the present invention described above and the cacao extract obtained by the production method of the present invention described above. Thus, the cacao-derived water extract (or cacao extract) according to the present invention can be effectively used as an antioxidant composition. Such antioxidant composition is also included in the technical scope of the present invention.

Polyphenol has various physiologically active functions such as active oxygen inhibition function, antiaging function, cancer prevention function, anti-stress function, arteriosclerosis prevention function and the like. Thus, the cacao-derived water extract of the present invention can be used as an active ingredient of active oxygen inhibitors, antiaging agents, cancer preventive or therapeutic agents, anti-stress agents, arteriosclerosis inhibitors and the like.

In addition, the cacao-derived water extract (or cacao extract) of the present invention contains a large amount of antioxidant components, and thus can be used as an active ingredient of food or drink (except for animals and plants themselves), pharmaceuticals, chemical products, cosmetics, and the like. As described above, the cacao extract of the present invention uses water as an extraction solvent; thus, it has less miscellaneous flavor components and has a better flavor as compared with the case of using an organic solvent such as ethanol or the like. Thus, the cacao-derived water extract (or cacao extract) of the present invention is preferably used as an active ingredient of foods and beverages (except animals and plants per se). Food or drink products (except for animals and plants themselves) containing the cacao water extract of the present invention are also included in the technical scope of the present invention.

Besides cacao extract, the food or drink of the present invention may contain well-known additives which can be contained in a food (for example, functional food). Examples of such additives include water, sugars, sugar alcohols, starch and processed starch, dietary fiber, milk, processed milk, soy milk, fruit juice, vegetable juice, fruits and vegetables and processed products thereof, proteins, peptides, amino acids, animal and plant herbal extracts, natural polymers (collagen, hyaluronic acid, chondroitin or the like), vitamins, minerals, thickeners, emulsifiers, preservatives, coloring agents, perfumes and the like. Specific examples of foods and beverages include beverages, cocoa, chocolate, coffee, gum, gummy, jelly, dairy products and the like. However, the form of the food or drink should not be particularly limited.

Note that the use of the cacao extract of the present invention should not be limited to use in foods and beverages. The antioxidant component contained in the cacao extract of the present invention can be used as an active ingredient of pharmaceuticals, chemical products, cosmetics and the like besides food or drink.

In a case of using the cacao extract of the present invention as pharmaceuticals, well-known additives which can be contained in pharmaceuticals may be contained in addition to the cacao extract. Examples of such additives include excipients, disintegrants, binders, fluidizing agents, corrigents, perfumes, colorants, sweeteners, solvents, oils and fats, thickeners, surfactants, gelling agents, stabilizers, preservatives, buffers, suspending agents, thickening agents and the like.

[Method for Extracting Polyphenols]

Next, a polyphenol extraction method according to the present invention will be described. The extraction method according to the present invention includes immersing the pulverized cacao raw material in water with a temperature within a range from 50° C. to 90° C. inclusive and a pH within a range from 2.0 to 5.0 inclusive, and extracting cacao-derived components. This extraction method makes it possible to efficiently extract polyphenols in water while suppressing the extraction amount of the miscellaneous flavor component to a low level.

As the cacao raw material used in the extraction method according to the present invention, the same cacao raw material as the above-described method for producing the cacao extract of the present invention can be used. Also, in the extraction method of the present invention, the temperature and pH of water used as an extraction solvent can be adjusted by the same method as the above-described method for producing cacao extract of the present invention. Also, regarding the extraction conditions in the extraction method of the present invention, the same conditions as those of the above-described cacao extract production method of the present invention can be employed.

It should be noted that each of embodiments disclosed herein is an example in all respects and it is not restrictive. The scope of the present invention is defined not by the above description but by the scope of the claims, and it is intended that all modifications within meaning and scope equivalent to the claims are included in the scope of the present invention.

### EXAMPLE

The present invention will now be described in more details with reference to examples. The following examples are merely illustrative, and do not limit the present invention.

### [Test 1]

In this Test 1, as an example of a method for producing the cacao extract of the present invention, an extraction test was conducted using water with 70° C. and pH 4.0. In the extraction test, the mass of cacao raw material used and the extraction period were appropriately changed. These extraction tests are referred to as Example 1-1 to Example 1-6. For comparison, a similar extraction test was carried out using water with a temperature of 10° C. and water with a temperature of 30° C. These extraction tests are referred to as Comparative Examples 1-1 to 1-3.

### <Method of Extraction Test>

### (A) Raw Material

In Test 1, the following were used as cacao raw materials. Unfermented bean derived cacao powder (total polyphenol amount: 70.6 mg/g, six kinds of low molecular proanthocyanidins: 36.1 mg/g, theobromine: 14.0 mg/g).

### (B) Method

A cacao extract was obtained from the cacao raw material according to the following procedure.
(1) Cacao powder was added to 1,000 g of ultrapure water and suspended. Here, the amount of cacao powder to be added was changed for each test to be one of 50 g, 80 g, 100 g, and 125 g.
(2) Anhydrous citric acid powder and saturated citric acid solution were added to the suspension, and the suspension was adjusted to pH 4.0.
(3) The suspension was heated to 70° C. and had been stirred for an hour for extraction. For comparison, a case in which the temperature was set to 10° C. and a case in which the temperature was set to 30° C. were conducted. For comparison, tests were also conducted when the extraction time was prolonged to 3 hours, 5 hours, and 16 hours.
(4) The suspension was cooled to 20° C.
(5) Each suspension was centrifuged, and the supernatants were recovered. The conditions for centrifugation for the above test were 3,500 g, 10 minutes, and 25° C.
(6) The supernatant recovered in the above (5) was filtered through a stainless steel mesh (mesh count 150 meshes, mesh size of about 100 μm).
(7) The filtrate was concentrated with a rotary evaporator so that the solid content of Brix (Bx) was about 25% to obtain a cacao extract.
(8) Component analysis was performed on the obtained cacao extract by the following method.

(Measurement of Total Polyphenol Content)

The total polyphenol content was measured by the Prussian blue method in the following procedure.
(1) The target sample was diluted with methanol to an appropriate concentration to obtain a 50% methanol solution. The methanol solution was then filtered through a 0.45 μm filter to obtain an analytical sample.
(2) 100 μl of the analytical sample was added to 50 ml of pure water, and the solution was stirred with a stirrer bar.
(3) 3 ml of a 0.1 M solution of ferric ammonium sulfate (III) dodecahydrate was added to the sample of (2), and the solution was stirred for 20 minutes to react.
(4) 3 ml of an 8 mM of potassium hexacyanoferrate (III) solution was added to the above sample of (3), and the solution was stirred for 20 minutes to react.
(5) After the reaction, the absorbance at 720 nm of the sample of (4) was measured with a spectrophotometer.
(6) A calibration curve was obtained from measured values using commercially available epicatechin in the same manner as above, and the total amount of polyphenol was calculated as epicatechin equivalent.

(Measurement of Amount of Low Molecular Proanthocyanidins)

The amount of low molecular proanthocyanidins was measured by the reverse phase HPLC method.
(1) The target sample was diluted with methanol to an appropriate concentration to obtain a 50% methanol solution. Subsequently, the methanol solution was filtered through a 0.45 μm filter to obtain an analytical sample.
(2) The HPLC analysis was performed on each analysis sample under the following conditions.
Column: Deverosil ODS-HG-5 (4.6×250 mm) (manufactured by Nomura Chemical Co., Ltd.)
Column oven temperature: 40° C.
Mobile phase: A 0.1% trifluoroacetic acid (TFA) aqueous solution
   B 0.1% TFA acetonitrile solution
Flow rate: 0.8 ml/min
Injection amount: 10 μl
Detector: UV detector (wavelength 280 nm)

Gradient conditions of mobile phases A and B are shown in Table 1 below.

TABLE 1

| | Flow | Mobile | Mobile |
|---|---|---|---|
| Time | rate | phase A | phase B |
| (min) | (mL/min) | (%) | (%) |
| 0 | 0.8 | 90 | 10 |
| 5 | 0.8 | 90 | 10 |
| 35 | 0.8 | 75 | 25 |
| 40 | 0.8 | 0 | 100 |
| 45 | 0.8 | 0 | 100 |

(3) A calibration curve was obtained from measured values using commercially available epicatechin and the amount of proanthocyanidins was calculated as epicatechin equivalent in the same manner as described above.

(Measurement of the Amount of theobromine)

The amount of theobromine was measured by the following procedure by the HPLC method.
(1) The target sample was diluted with water to an appropriate concentration to obtain an aqueous solution. Subsequently, the aqueous solution was filtered through a 0.45 μm filter to obtain an analytical sample.
(2) The HPLC analysis was performed on each analysis sample under the following conditions.
Column: ODS-80TM (4.6×250 mm) (manufactured by Tosoh Corporation)
Column oven temperature: 40° C.
Mobile phase: A 0.01 N phosphate buffer, pH 3.5
B 0.1% methanol
Flow rate: 1.0 ml/min
Injection amount: 10 μl
Detector: UV detector (wavelength 280 nm)

The gradient conditions of mobile phases A and B are shown in Table 2 below.

TABLE 2

| | Flow | Mobile | Mobile |
|---|---|---|---|
| Time | rate | phase A | phase B |
| (min) | (mL/min) | (%) | (%) |
| 0 | 1.0 | 78 | 22 |
| 20 | 1.0 | 78 | 22 |
| 22 | 1.0 | 0 | 100 |
| 26 | 1.0 | 0 | 100 |
| 30 | 1.0 | 78 | 22 |

(3) In the same manner as above, a calibration curve was obtained from measured values using commercially available theobromine to determine the amount of theobromine in the sample.

The results of the above tests are shown in Table 3 below. In Table 3, “total poly” represents the total amount of polyphenols contained in the extract. In addition, “six kinds of Pr” refers to the total amount of six kinds of proanthocyanidins of catechin (ca), epicatechin (cp), procyanidin B2 (b2), procyanidin B5 (b5), procyanidin C1 (c1), and cinnamtannin A2 (A2).

TABLE 3

| | cacao raw | Water | Extraction | | Concentration (mg/g) | | | Extraction rate (%) | | |
|---|---|---|---|---|---|---|---|---|---|---|
| | material | temperature | time | | Total | Six kinds | | Total | Six kinds | |
| No. | (g) | (° C.) | (hour) | Brix | poly | of Pr6 | Theobromine | poly | of Pr6 | Theobromine |
| Example 1-1 | 50 | 70 | 1 | 26.2 | 27.4 | 15.7 | 8.2 | 52.7 | 52.7 | 63.0 |
| Example 1-2 | 80 | 70 | 1 | 25.4 | 23.8 | 15.0 | 7.8 | 39.4 | 43.4 | 52.1 |
| Example 1-3 | 80 | 70 | 3 | 25.8 | 24.4 | 13.7 | 7.6 | 41.4 | 40.6 | 52.1 |
| Example 1-4 | 80 | 70 | 5 | 26.7 | 23.8 | 12.8 | 7.6 | 38.1 | 35.7 | 48.5 |
| Example 1-5 | 100 | 70 | 1 | 28.1 | 23.9 | 13.5 | 7.6 | 33.4 | 33.0 | 42.3 |
| Example 1-6 | 125 | 70 | 1 | 24.5 | 21.2 | 11.9 | 6.8 | 30.0 | 29.5 | 38.9 |
| Comparative | 80 | 10 | 1 | 30.5 | 30.7 | 18.4 | 9.8 | 34.5 | 36.1 | 44.3 |
| Example 1-1 | | | | | | | | | | |
| Comparative | 80 | 30 | 1 | 26.2 | 27.8 | 18.6 | 10.1 | 32.8 | 38.2 | 47.8 |
| Example 1-2 | | | | | | | | | | |
| Comparative | 80 | 30 | 16 | 24.8 | 20.3 | 13.0 | 8.7 | 29.4 | 33.0 | 50.4 |
| Example 1-3 | | | | | | | | | | |

It has been confirmed from the results shown in Table 3 that in a case where the cacao raw material is 80 g, setting the temperature of the water is set to 70° C. enhances the extraction rate of polyphenols and proanthocyanidins as compared with a case where the water temperature is set to 10° C. and 30° C. In Examples 1-1 to 1-6, it has been confirmed that the concentration of theobromine can be set to an appropriate amount within a range from 5 mg/g to 10 mg/g inclusive. Although not shown in the table, it has been confirmed that even when 100 g of the cacao raw material is used, the same result as the above example can be obtained.

In addition, it has been confirmed that the extraction rate of polyphenols and proanthocyanidins can be enhanced by using more water for the cacao raw material. It has been confirmed that the extraction rate of proanthocyanidins can be enhanced by setting the extraction time to one hour, as compared with a case where longer extraction time is set. This is considered to be due to progression of denaturation, decomposition, and the like of proanthocyanidins caused by prolonging the extraction time.

From the above, it can be ascertained that the total polyphenol amount and proanthocyanidins can be extracted with high efficiency by obtaining the cacao extract under the extraction conditions of Example 1-1 to Example 1-6. Note that the extracts of cacao extracted under the extraction conditions of Example 1-1 to Example 1-6 are extremely highly likely to be included in the technical scope of the invention according to claim 1 (invention of matter) in consideration of the extraction conditions.

### [Test 2]

In Test 2, with the method for producing cacao extract of the present invention, extraction test was conducted by changing the temperature and pH of water. Subsequently, component analysis was performed on the obtained cacao extract.

<Method of Extraction Test>

### (A) Raw Material

In this test 2, the following were used as cacao raw materials.

Unfermented bean derived cacao powder (total polyphenol content: 70.1 mg/g, six kinds of low molecular proanthocyanidins: 31.7 mg/g)

### (B) Method

A cacao extract was obtained from the cacao raw material through the following procedure.
(1) 85 ml of ultrapure water was poured into a 200 ml Erlenmeyer flask with 1.0 g of cacao powder, and suspended.
(2) Citric acid (anhydrous citric acid powder and saturated citric acid solution) was added to the suspension of (1), and the pH was adjusted to 2.0, 3.5 and 5.0 individually.
(3) The temperature of the suspension of (2) was heated to 50° C., 70° C., and 90° C., and stirred for 1 hour for extraction.
(4) After each suspension of (3) was cooled, it was centrifuged and the supernatants were retrieved to obtain a cacao extract. The condition of centrifugation here was 10,000 g, 10 minutes, 25° C.
(5) Component analysis was performed on the obtained cacao extract by the following method. In the component analysis here, each content (μg/ml) of the above six proanthocyanidins and each content (μg/ml) of monomeric to octameric proanthocyanidins was measured.

(Measurement of Content of Six Kinds of Proanthocyanidins)

The contents of six kinds of proanthocyanidins were each measured by the reversed phase HPLC method in the same manner as the method for measuring the amount of low molecular proanthocyanidins in Test 2 described above. A calibration curve was obtained from measured values using commercially available epicatechin and each amount of proanthocyanidins was calculated as epicatechin equivalent.

(Measurement of Amounts of Monomeric to Octameric Proanthocyanidin Polymers)

The amounts of proanthocyanidin polymers were measured by the normal phase HPLC according to the following procedure.
(1) The target sample was diluted with methanol to an appropriate concentration to obtain a 50% methanol solution. Subsequently, the methanol solution was filtered through a 0.45 μm filter to obtain an analytical sample.
(2) The HPLC analysis was performed on each analysis sample under the following conditions.

Column: Deverosil Diol-5 (4.6×250 mm) (manufactured by Nomura Chemical Co., Ltd.)
Column oven temperature: 40° C.
Mobile phase: A methanol: pure water: acetic acid=95:3:2
   B Acetonitrile: acetic acid=49:1
Flow rate: 0.8 ml/min
Injection amount: 10 μl
Detector: UV detector (wavelength 280 nm)

The gradient conditions for mobile phases A and B are shown in Table 4 below.

TABLE 4

| | Flow | Mobile | Mobile |
|---|---|---|---|
| Time | rate | phase A | phase B |
| (min) | (mL/min) | (%) | (%) |
| 0 | 0.8 | 0 | 100 |
| 35 | 0.8 | 40 | 60 |
| 40 | 0.8 | 40 | 60 |

(3) A calibration curve was obtained from measured values using commercially available epicatechin in the same manner as above, and each amount of proanthocyanidins whose polymerization degrees are one to eight was calculated as an epicatechin equivalent.

The results of the above tests are shown in the following Tables 5 to 7. Table 5 shows the analysis results on the six kinds of proanthocyanidins. In Table 5, “ca” represents catechin, “ep” represents epicatechin, “b2” represents procyanidin B2, “b5” represents procyanidin B5, “c1” represents procyanidin C1, and “A2” represents cinnamtannin A2. Table 5 shows the contents (μg/ml) and mass ratios of the six proanthocyanidins

TABLE 5

| | | ca | | ep | | b2 | | b5 | | c1 | | A2 | | |
|---|---|---|---|---|---|---|---|---|---|---|---|---|---|---|
| Temp. | | | mass | | mass | | mass | | mass | | mass | | mass | |
| (° C.) | pH | content | ratio | content | ratio | content | ratio | content | ratio | content | ratio | content | ratio | b2/ca |
| 50 | 5.0 | 4.5 | 1.96 | 103.5 | 45.04 | 46.8 | 20.37 | 9.0 | 3.92 | 42.8 | 18.62 | 23.2 | 10.10 | 10.40 |
| | 3.5 | 5.8 | 1.88 | 132.4 | 42.96 | 68.6 | 22.26 | 14.3 | 4.64 | 57.8 | 18.75 | 29.3 | 9.51 | 11.93 |
| | 2.0 | 5.0 | 1.74 | 131.3 | 38.15 | 101.4 | 29.46 | 14.9 | 4.33 | 65.6 | 19.06 | 25.0 | 7.26 | 15.90 |
| 70 | 5.0 | 6.4 | 2.42 | 118.0 | 44.60 | 55.2 | 20.86 | 10.6 | 4.01 | 49.8 | 18.82 | 24.6 | 9.30 | 8.63 |
| | 3.5 | 6.0 | 1.90 | 129.5 | 41.03 | 79.6 | 25.22 | 13.7 | 4.34 | 58.8 | 18.63 | 28.0 | 8.87 | 13.27 |
| | 2.0 | 6.3 | 1.81 | 122.7 | 35.19 | 124.2 | 35.62 | 14.7 | 4.22 | 61.0 | 17.49 | 19.8 | 5.68 | 19.71 |
| 90 | 5.0 | 22.5 | 8.20 | 114.6 | 41.76 | 60.9 | 22.19 | 7.9 | 2.88 | 43.2 | 15.74 | 25.3 | 9.22 | 2.71 |
| | 3.5 | 7.3 | 2.25 | 124.5 | 38.32 | 109.3 | 33.64 | 13.7 | 4.22 | 49.7 | 15.30 | 20.4 | 6.28 | 14.97 |
| | 2.0 | 12.3 | 3.90 | 121.3 | 38.50 | 116.9 | 37.10 | 13.4 | 4.25 | 39.5 | 12.54 | 11.7 | 3.71 | 9.50 |

Table 6 and Table 7 show the analysis results on monomeric to octameric proanthocyanidins Table 6 shows each content (μg/ml) of monomeric to octameric proanthocyanidins Also, Table 7 shows each mass ratio of monomeric to octameric proanthocyanidins Note that Table 7 also shows the ratio of the total mass of dimeric to tetrameric proanthocyanidins to the total mass of proanthocyanidins (dimer to tetramer/monomer to octamer (%)).

TABLE 6

| Temp. | | | | | | | | | |
|---|---|---|---|---|---|---|---|---|---|
| (° C.) | pH | monomer | dimer | trimer | tetramer | pentamer | hexamer | heptamer | octamer |
| 50 | 5.0 | 118.9 | 50.1 | 35.0 | 28.1 | 16.8 | 10.2 | 2.3 | 0.0 |
| | 3.5 | 147.1 | 73.2 | 54.9 | 41.8 | 23.2 | 14.4 | 3.6 | 0.0 |
| | 2.0 | 145.5 | 106.1 | 70.4 | 44.7 | 24.4 | 12.0 | 4.5 | 0.7 |
| 70 | 5.0 | 135.0 | 60.9 | 43.0 | 33.4 | 18.3 | 14.5 | 1.3 | 0.0 |
| | 3.5 | 144.7 | 87.2 | 61.3 | 46.7 | 25.2 | 17.0 | 0.0 | 0.0 |
| | 2.0 | 139.0 | 129.9 | 71.3 | 47.0 | 26.1 | 12.8 | 0.0 | 0.0 |
| 90 | 5.0 | 148.9 | 71.3 | 52.9 | 39.0 | 21.4 | 11.1 | 0.0 | 0.0 |
| | 3.5 | 137.5 | 113.4 | 66.7 | 44.4 | 23.4 | 9.6 | 0.0 | 0.0 |
| | 2.0 | 136.5 | 123.2 | 65.7 | 52.2 | 25.3 | 18.7 | 0.0 | 0.0 |

TABLE 7

| | | | | | | | | | | dimer to |
|---|---|---|---|---|---|---|---|---|---|---|
| | | | | | | | | | | tetramer/ |
| Temp. | | | | | | | | | | monomer to |
| (° C.) | pH | monomer | dimer | trimer | tetramer | pentamer | hexamer | heptamer | octamer | octamer (%) |
| 50 | 5.0 | 45.49 | 19.17 | 13.39 | 10.75 | 6.43 | 3.90 | 0.88 | 0.00 | 43.31 |
| | 3.5 | 41.07 | 20.44 | 15.33 | 11.67 | 6.48 | 4.02 | 1.01 | 0.00 | 47.44 |
| | 2.0 | 35.64 | 25.99 | 17.24 | 10.85 | 5.98 | 2.94 | 1.10 | 0.17 | 54.18 |
| 70 | 5.0 | 44.08 | 19.88 | 14.03 | 10.90 | 5.97 | 4.73 | 0.42 | 0.00 | 44.81 |
| | 3.5 | 37.87 | 22.82 | 16.04 | 12.22 | 6.60 | 4.45 | 0.00 | 0.00 | 51.08 |
| | 2.0 | 32.62 | 30.49 | 16.73 | 11.03 | 6.13 | 3.00 | 0.00 | 0.00 | 58.25 |
| 90 | 5.0 | 43.21 | 20.69 | 15.35 | 11.32 | 6.21 | 3.22 | 0.00 | 0.00 | 47.38 |
| | 3.5 | 34.81 | 28.71 | 16.89 | 11.24 | 5.92 | 2.43 | 0.00 | 0.00 | 56.84 |
| | 2.0 | 32.38 | 29.22 | 15.58 | 12.38 | 6.00 | 4.44 | 0.00 | 0.00 | 57.18 |

From the results shown in Table 5, it has been confirmed that producing a cacao extract using the method for producing a cacao extract of the present invention allows the ratio (b2/ca) of the mass of procyanidin B2 (b2) to the mass of catechin (ca) to be within an appropriate range (for example, within a range from 5 to 20 inclusive).

From the results shown in Tables 6 and 7, it has been confirmed that producing a cacao extract using the method for producing cacao extract of the present invention allows the ratio of the total mass of dimeric to tetrameric proanthocyanidins to the total mass of polyphenols to be 40% or more. Also, it has been confirmed that setting the temperature of water used for extraction to 70° C. or higher and pH of the water to 4.0 or lower allows the ratio of the total mass of dimeric to tetrameric proanthocyanidins to the total mass of polyphenols to be 51% or more.

### [Test 3]

In this Test 3, the type of solvent used for extracting polyphenol from the cacao raw material was changed, and it was investigated whether the amount of extracting proanthocyanidins differed depending on types of solvents.

<Method of Extraction Test>

### (A) Raw Material

In this Test 3, the following materials were used as cacao raw materials.

Unfermented bean derived cacao powder (low molecular proanthocyanidins: 38.9 mg/g)

### (B) Solvent

In this Test 3, the following four types of extraction solvents were used.

### EXAMPLE 3-1

### Water Adjusted to be with a Temperature of 70° C. and a pH of 3.5

### EXAMPLE 3-2

### Water Adjusted to be with a Temperature of 70° C. and a pH of 3.5, Extraction Step 2 Times

### COMPARATIVE EXAMPLE 3-1

### 70% Ethanol (Temperature 70° C., pH not Adjusted)

### COMPARATIVE EXAMPLE 3-2

### Water with a Temperature of 70° C. and with its pH Unadjusted

### (C) Method

A cacao extract was obtained from the cacao raw material according to the following procedure.
(1) Cacao powder was added to each solvent and suspended. Here, the mass ratio of the cacao powder to the solvent was set to 1:50.
(2) In Examples 3-1 and 3-2, citric acid (anhydrous citric acid powder and saturated citric acid solution) was added to the suspension of (1) to adjust the pH to 3.5.
(3) The temperature of the suspension of (2) was heated to 70° C. and stirred for 1 hour for extraction.
(4) After each suspension of (3) was cooled, it was centrifuged and the supernatants were retrieved to obtain a cacao extract. The condition for centrifugation here was 3,500 g, 10 minutes, 25° C.
(5) With respect to the obtained cacao extract, component analysis was performed in the same manner as in Test 2, and the content of proanthocyanidins was measured.

In Example 3-2, a second extraction step was performed between (4) and (5). In the second extraction step, the same amount of water as the water added first was added to the residue retrieved in the first solid-liquid separation step, and the suspension was suspended for 10 minutes. Subsequently, solid-liquid separation was performed by the same method as in the first method, and the supernatant was retrieved, mixed with the supernatant in the first extraction step, and subjected to component analysis.

The results of the above tests are shown in Table 8 below. Table 8 shows the ratio (%) of the mass of proanthocyanidins in the cacao extract to the mass of proanthocyanidins in the raw cacao powder. In Table 8, this percentage (%) is expressed as “recovery rate (%) of proanthocyanidins”.

TABLE 8

| | Recovery rate (%) |
|---|---|
| | of proanthocyanidins |
| Example 3-1 | 56.2 |
| Example 3-2 | 63.3 |
| Comparative Example 3-1 | 72.1 |
| Comparative Example 3-2 | 42.1 |

Based on the results of the above tests, it has been confirmed that producing the cacao extract using the method for producing the cacao extract of the present invention makes it possible to extract proanthocyanidins with high efficiency as compared with a case without pH adjustment. In addition, it has been confirmed that performing the extraction step a plurality of times makes it possible to extract proanthocyanidins to a degree comparable to that obtained by performing extraction with ethanol.

### [Test 4]

In Test 4, flavors were evaluated on the cacao extract obtained in Example 3-1 (acid hot extract) and Comparative Example 3-1 (ethanol extract). Regarding each cacao extract, the concentration was adjusted using a diluent or the like so that low molecular proanthocyanidins are contained with a concentration of 100 mg/100 ml. Sucrose was then added to each cacao extract so as to be 4% (w/w).

Evaluation of flavor was carried out by ten specialty sensory panelists who passed the five-flavor discrimination test specified in the company of the present applicant. Ten panelists tasted each cacao extract and evaluated the following items in five stages.

(Evaluation Item)
Bitterness: 5 (strong) to 1 (weak)
Astringency: 5 (strong) to 1 (weak)
Miscellaneous flavor (Impure taste): 5 (strong) to 1 (weak)
Fruity sense: 5 (strong) to 1 (weak)

The results of the above tests are shown in Table 9 below.

TABLE 9

| | Example 3-1 | Comparative |
|---|---|---|
| | acid hot | Example 3-1 |
| | water | ethanol |
| | extract | extract |
| Bitterness | 2.5 | 4.0 |
| Astringency | 2.8 | 4.4 |
| Miscellaneous flavor | 2.7 | 3.8 |
| Fruity sense | 3.3 | 2.5 |

From the above results, it has been confirmed that in the cacao extract obtained by the method for producing the cacao extract of the present invention, all of the bitterness, astringency and miscellaneous flavor (impure taste) are reduced as compared with the cacao extract extracted using ethanol. In addition, it has been confirmed that the cacao extract obtained by the method for producing cacao extract of the present invention has a better flavor as compared with the cacao extract extracted with ethanol. From this result, it has been ascertained that the cacao extract obtained by the method for producing the cacao extract of the present invention presents a comprehensively good evaluation regarding the taste as compared with the cacao extract extracted using ethanol.

### [Test 5]

<Method of Extraction Test>

### (A) Raw Material

In Test 5, the following cacao beans were used as a raw material.

Ecuadorian cacao beans (total polyphenol amount: 37.0 mg/g, six kinds of low molecular proanthocyanidins: 5 0 mg/g).

Note that the amount of active ingredient in Ecuadorian cacao beans used in Test 5 is lower than that of unfermented beans used in Test 1.

### (B) Method

(1) The raw cacao beans were roughly crushed using pliers. The particle size distribution of the crushed cacao beans was measured by sieving in a stepwise manner using a stainless steel mesh with each of various openings. The results are shown in Table 10.

TABLE 10

| Particle size | Ratio |
|---|---|
| (mm) | (weight ratio %) |
| 4.75~2.0 | 78.7 |
| 2.0~1.18 | 12.5 |
| 1.18~0.5 | 6.5 |
| 0.5~0.21 | 1.9 |
| 0.21 or less | 0.4 |

(2) Various amounts of ultrapure water were individually added to 50 g of roughly crushed cacao beans and suspended. The weight of the added ultrapure water was set to be 4 times, 6 times, 8 times, 10 times, or 12 times the weight of the cacao beans.
(3) Anhydrous citric acid powder and saturated citric acid solution were added to the suspension, and the suspension was adjusted to be with pH 4.0.
(4) The mixture was heated to 70° C. and stirred for 1 hour for extraction.
(5) The suspension was cooled to 20° C.
(6) Each suspension was filtered through a stainless steel mesh (mesh count 20 meshes, mesh size of about 850 μm) to remove solid contents with large particle sizes.
(7) Next, each suspension was filtered through a stainless steel mesh (mesh count 50 meshes, mesh size of about 300 μm) to remove solid contents with moderate particle sizes.
(8) Next, each suspension was filtered through a stainless steel mesh (mesh count 150 meshes, mesh size of about 106 μm) to remove solid contents with small particle sizes.
(9) The filtrate was concentrated with a rotary evaporator so that the Brix (Bx) solid content was about 25%, thereby obtaining a cacao extract.
(10) With respect to the obtained cacao extract, the total amount of polyphenols and the amount of low molecular proanthocyanidins were measured in the same manner as in Test 1. The results are shown in Table 11 below. In Table 11, “total poly” represents the total amount of polyphenols contained in the extract. In addition, “six kinds of Pr6” refers to the total amount of six kinds of proanthocyanidins of catechin (ca), epicatechin (ep), procyanidin B2 (b2), procyanidin B5 (b5), procyanidin C1 (c1), cinnamtannin A2 (A2). Note that in Table 11, the total amount of polyphenols and the total amount of proanthocyanidins of species are shown as the active ingredient recovery rate (%).

TABLE 11

| | Active ingredient | |
|---|---|---|
| Additive amount | recovery rate (%) | |
| of water | Total | Six kinds |
| (n of n times amount) | poly | of Pr6 |
| 4 | 13.1 | 24.7 |
| 6 | 16.0 | 31.7 |
| 8 | 17.6 | 35.8 |
| 10 | 18.6 | 38.2 |
| 12 | 19.3 | 39.8 |

From the above results, it has been confirmed that in the cacao extract obtained by using the roughly crushed cacao beans, the extraction amount of low molecular proanthocyanidins relative to the total amount of polyphenols increases as compared with the cacao extract obtained using the powdery cacao raw material used in Test 1.

### [Test 6]

<Method of Extraction Test>

### (A) Raw Material

In Test 6, the following cacao beans were used as a raw materials.
Unfermented cacao beans from Vietnam (total amount of polyphenols: 59.6 mg/g, six kinds of low molecular proanthocyanidins: 20.1 mg/g).

### (B) Method

(1) The raw cacao beans were roughly crushed using pliers in the same manner as in Test 5.
(2) Various amounts of ultrapure water were individually added to 50 g of roughly crushed cacao beans, and suspended. The weight of the added ultrapure water was set to be 6 times, 8 times, or 12 times the weight of the cacao beans.
(3) Anhydrous citric acid powder and saturated citric acid solution were added to the suspension, and the suspension was adjusted to be with pH 4.0.
(4) The mixture was heated to 70° C. and stirred for an hour for extraction.
(5) The suspension was cooled to 20° C.
(6) Each suspension was filtered through a stainless steel mesh (mesh count 20 meshes, mesh size of about 850 μm) to remove solid contents of large particle sizes.
(7) Next, each suspension was filtered through a stainless steel mesh (mesh count 50 meshes, mesh size of about 300 μm) to remove solid contents of moderate particle sizes.
(8) Next, each suspension was filtered through a stainless steel mesh (mesh count 150 meshes, mesh size of about 106 μm) to remove solid contents with small particle sizes.
(9) The filtrate was concentrated with a rotary evaporator so that the Brix (Bx) solid content was about 25%, thereby obtaining a cacao extract.
(10) With respect to the obtained cacao extract, the total amount of polyphenols and the amount of low molecular proanthocyanidins were measured in the same manner as in Test 1. The results are shown in Table 12 below. In Table 12, “total poly” represents the total amount of polyphenols (concentration (mg/g) and recovery rate (%)) contained in the extract. In addition, “six kinds of Pr6” refers to the total amount of six kinds of proanthocyanidins (concentration (mg/g) and recovery rate (%)) of catechin (ca), epicatechin (ep), procyanidin B2 (b2), procyanidin B5 (b5), procyanidin C1 (c1), cinnamtannin A2 (A2). For comparison, Table 12 additionally shows that the total amount of polyphenols contained in the raw cacao beans and the total amount of the six kinds of proanthocyanidins

TABLE 12

| Additive | | | | | | |
|---|---|---|---|---|---|---|
| amount | | | Concentration | | Recovery rate | |
| of water | | | (mg/g) | | (%) | |
| (n of n times | | Mass | Total | Six kinds | Total | Six kinds |
| amount) | | (g) | poly | of Pr6 | poly | of Pr6 |
| 6 times | Raw material | 50.0 | 59.6 | 20.1 | — | — |
| | bean | | | | | |
| | Concentrated | 25.8 | 28.4 | 16.7 | 24.6 | 42.7 |
| | extract | | | | | |
| 8 times | Raw material | 50.0 | 59.6 | 20.1 | — | — |
| | bean | | | | | |
| | Concentrated | 28.3 | 31.0 | 15.2 | 29.5 | 42.9 |
| | extract | | | | | |
| 12 times | Raw material | 50.0 | 59.6 | 20.1 | — | — |
| | bean | | | | | |
| | Concentrated | 20.3 | 41.5 | 22.0 | 28.2 | 44.3 |
| | extract | | | | | |

From the above results, it has been confirmed that low molecular proanthocyanidins are contained, at a high content percentage, in the cacao extract obtained by using the roughly crushed cacao beans, as compared with the cacao beans as a raw material. In addition, it has been confirmed that in the cacao extract obtained by using the roughly crushed cacao beans, low molecular proanthocyanidins can be extracted at a high recovery rate as compared with the cacao extract obtained by using powdery cacao raw material used in Test 1.

1. A cacao-derived water extract containing polyphenol, wherein
the polyphenol includes proanthocyanidins, and
a ratio of a total mass of dimeric proanthocyanidins, trimeric proanthocyanidins and tetrameric proanthocyanidins to a total mass of monomeric to octameric proanthocyanidins is 40% or more.

2. The cacao-derived water extract according to claim 1, wherein a ratio of a total mass of dimeric proanthocyanidins, trimeric proanthocyanidins and tetrameric proanthocyanidins to a total mass of monomeric to octameric proanthocyanidins is 51% or more.

3. The cacao-derived water extract according to claim 1,
wherein procyanidin B2 and catechin are contained as the polyphenol, and
the ratio of a mass of the procyanidin B2 to a mass of the catechin is within a range from 5 to 20 inclusive.

4. The cacao-derived water extract according to claim 1, wherein the polyphenol contains no octameric proanthocyanidins.

5. The cacao-derived water extract according to claim 1, the cacao-derived water extract including theobromine with a concentration within a range from 5 mg/g to 20 mg/g inclusive.

6. A food or drink comprising the cacao-derived water extract according to claim 1.

7. A method for producing a cacao extract, comprising:
providing a cacao or a cacao processed product as a raw material; and
extracting a cacao-derived component using water adjusted to have a temperature within a range from 50° C. to 90° C. inclusive and a pH within a range from 2.0 to 5.0 inclusive.

8. The method for producing a cacao extract according to claim 7, further comprising:
adjusting the pH of the water to a pH that is greater than 4.0 and lower than or equal to 5.0.

9. The method for producing a cacao extract according to claim 7, wherein the raw material is roughly crushed cacao beans.

10. A cacao extract, using a cacao or a cacao processed product as a raw material, wherein the cacao extract is extracted using water having a temperature within a range from 50° C. to 90° C. inclusive and a pH within a range from 2.0 to 5.0 inclusive.

11. A method of extracting polyphenol, comprising:
immersing crushed cacao raw material in water having a temperature within a range from 50° C. to 90° C. inclusive and a pH within a range from 2.0 to 5.0 inclusive to extract a cacao-derived component in the water, and
wherein the method extracts polyphenol in the water while maintaining an extracted amount of miscellaneous flavor components to a low level.
